(19) United States
(12) Patent Application Publication
(10) Pub. No.: US 2022/0090090 A1
(43) Pub. Date: Mar. 24, 2022

(54) A METHOD FOR ASSEMBLING CIRCULAR AND LINEAR DNA MOLECULES IN AN ORDERED MANNER

(71) Applicant: UNIVERSITY OF MARLYLAND, BALTIMORE COUNTY, Baltimon, MD (US)

(72) Inventors: CHARLES J. BIEBERICH, BROOKEVILLE, MD (US); XIANG LI, BALTIMORE, MD (US)

(21) Appl. No.: 17/419,893

(22) PCT Filed: Jan. 15, 2020
(86) PCT No.: PCT/US2020/013587

§ 371 Date: Jun. 30, 2021

Related U.S. Application Data
(60) Provisional application No. 62/792,532, filed on Jan. 15, 2019.

(51) Int. Cl. C12N 15/66; C12N 15/64

(52) U.S. Cl. CPC C12N 15/66; C12N 9/22; C12N 15/64

(57) ABSTRACT

The present invention relates to a method of assembling circular and linear DNA molecules, more specifically, the present invention provides for a homology-based, one-tube assembly method including a circular DNA vector and at least one restriction enzyme without prior linearization of such a circular DNA vector.

### CROSS REFERENCE TO RELATED APPLICATION

This application claims priority to U.S. Provisional Patent Application No. 62/792532, filed on Jan. 15, 2019, the contents of which are hereby incorporated by reference herein for all purposes.

### BACKGROUND OF THE INVENTION

### Field of the Invention

The present invention relates to a method of assembling circular and linear DNA molecules, more specifically, the present invention provides for a homology-based, one-tube assembly method including a non-linearized circular DNA vector and at least one restriction enzyme for assembling the circular and linear DNA molecules.

### Related Art

Cloning a specific gene into a circular plasmid vector is often the first step in studying gene functions. Before homology-based cloning strategies were developed, gene cloning has been achieved by digesting the target gene and the vector with restriction endonucleases followed by ligating them together by using a DNA ligase. This process can be technically challenging especially when the target gene to be cloned is generated by PCR. The main hurdle for cloning PCR products is the often low efficiency of restriction enzyme digestion of PCR products.

Homology-based cloning strategies greatly increase the efficiency of cloning PCR products. Multiple homology-based strategies have been described. The “Gibson Assembly” method starts with linearized vectors and uses three types of enzymes in the same reaction: T5 exonuclease, Phusion DNA polymerase and Taq ligase [1]. The “In-Fusion” method uses a similar strategy, however no Taq ligase is used [2]. These strategies can assemble multiple DNA fragments into one plasmid DNA vector with high efficiency. Another method was recently described in which only T5 exonuclease is required to perform the assembly [3].

However, in all of the strategies described above, linearization of the circular plasmid DNA prior to the assembly reaction is required. Further, in most systems, the linearized vector needs to be purified by agarose gel electrophoresis before assembly. This process is time consuming, can be technically challenging, and adds cost. Thus, it would be advantageous to provide a method of assembling both circular and linear DNA to overcome the short comings of the prior art.

### SUMMARY OF THE INVENTION

The present invention provides a method to assemble circular or linear DNA molecules wherein a circular DNA vector is directly assembled with a linear nucleotide product in one step and in one reaction vessel.

In aspect the present invention provides for a one pot method to prepare a circular or linear DNA molecule for use in preparing a nucleotide end-product, the method comprising:
providing a reaction vessel, a combination of a circular DNA vector and a linearized target DNA molecule with regions of sequence having homology to the vector on both ends;
introducing into the reaction vessel at essentially the same time the circular DNA vector and the amplified linearized target DNA molecule and at least one restriction enzyme into the reaction vessel in an amount to linearize the circular DNA vector;
adding to the reaction vessel a buffering solution, wherein the buffering solution comprises at least a DNA polymerase, a 5′-3′ exonuclease, a buffering agent and optionally a DNA ligase;
incubating the circular DNA vector, the linearized target DNA molecule and the buffering solution for a sufficient time and temperature for linearization of the circular DNA vector and joining the amplified linearized target DNA molecule and the linearized circular DNA vector for production of the circularized or linearized DNA molecule, wherein the nucleotide end-product is selected from the group consisting of circular or linear DNA molecule, circular or linear RNA molecule or a protein encoded by the circularized or linearized DNA molecule through host cell production.

In preparation for the combination, the linearized target DNA molecule, can be amplified and then the linearized target DNA molecule is ready for the combination. Further, the DNA target molecule may include a single strand of nucleotides on both the 5′ and 3′ end corresponding to the DNA nucleotide single strands on the linearized circular DNA vector caused by the specific restriction enzyme used in the cutting process within the one pot system. Thus, the target DNA target molecule can be hybridized to the overlapping complementary single stranded DNA region on linearize circular DNA molecule once the circular DNA vector is linearized.

The linearized target DNA molecule may in some embodiments comprise multiple DNA fragments that are combined and wherein each such DNA fragment will include overlapping single stranded DNA ends that overlaps with the single stranded DNA ends of the next fragment and then the ends of the combination of fragments will overlap or have homology with the end sequences of the cut circular vector. DNA polymerases that work in the methods of joining such fragments are those having intrinsic exonuclease activity and are capable of performing the DNA joining reaction of the invention. Such polymerases have the ability to join two linear DNA molecules having ends with complementary nucleotide sequences. The DNA polymerases of the invention are either commercially available or may be prepared using standard recombinant DNA technology. The DNA polymerases useful in the joining the fragments have intrinsic 3′-5′ exonuclease activity or 5′-3′ exonuclease activity.

Importantly, the present invention has demonstrated the feasibility of this method with or without the use of a ligase in the system. The DNA ligase is a thermostable DNA ligase, such as Taq DNA ligase (New England Biolabs), 9N DNA ligase (New England Biolabs) or Ampligase (Illumina, San Diego, Calif.), preferably Taq ligase is used.

Any DNA polymerase enzyme can be used for the polymerase cycling assembly reaction in a method of the present invention. Preferably, the DNA polymerase is a high-fidelity DNA polymerase, meaning that the DNA polymerase has a proof-reading function such that the probability of introducing a sequence error into the resulting, intact nucleic acid molecule is low. Examples of DNA polymerases suitable for the polymerase cycling assembly reaction include, but are not limited to Phusion polymerase, platinum Taq DNA polymerase High Fidelity (Invitrogen), Pfu DNA polymerase, etc. Preferably, the DNA polymerase used in the PCR is a thermostable, high-fidelity DNA polymerase, such as Phusion DNA polymerase (New England Biolabs, Ipswich, Mass.).

In the present invention, the buffering solution comprises at least a crowding agent such as a PEG molecule, and other components including but not limited to components selected from a group consisting of potassium acetate, magnesium acetate, bovine serum albumin, dNTPs, and buffer agents, such as a Tris buffering agent. In one embodiment the buffering solution comprises 2% to 10% of PEG8000, 50 mM to about 150 mM of Tris-Acetate, a pH 6.5 to 8.5, about 0.09 mM to about 0.4 mM dNTPs, about 25 mM to about 75 mM of potassium acetate, about 10 mM to about 40 mM of magnesium acetate and about 50 mg/ml to about 150 mg/ml Bovine Serum Albumin. More preferably the buffer solution comprises about 5% PEG8000, about 100 mM Tris-Acetate, a pH 8, about 0.2 mM dNTPs, about 50 mM of potassium acetate, about 20 mM of magnesium acetate and about 100 mg/ml Bovine Serum Albumin.

The incubating time is preferably divided into at least two different time periods and temperature regimes to linearize the circular plasmid and produce the circularized or linearized DNA molecule. For example, the first time period and temperature may be from about 32° C. to about 40° C. for about 10 minutes to about 20 minutes and more preferably with a temperature of about 37° C. for about 15 minutes. The next period and temperature are from about 45° C. to about 55° C. for about 10 minutes to about 20 minutes and more preferably with a temperature of about 50° C. for about 15 minutes. Notably other temperatures and time periods have been found effective such as 50° C. for 60 minutes; 37° C. for 15 minutes+50° C. for 45 minutes and 37° C. for 30 minutes +50° C. for 30 minutes

In the present invention, at least one restriction enzyme is used and in some situations a combination of restriction enzymes is possible such as a combination of BamHI and SalI. It has also been found that EcoRI, Pstl, and HindIII work efficiently in the present invention and in the preferred buffer. Further is it believed restriction enzymes (endonucleases) can include those that produce blunt ends (e.g., Smal, Stul, ScaI, EcoRV) or 3′ overhangs (e.g., Notl, BamHI, EcoRI, Spel, Xbal, HaeIII, Taql, AluI) In some situations, other restriction endonucleases that produce 5′ overhangs can also be used.

In another aspect, the present invention provides for a one pot method to prepare a circular or linear DNA molecule, the method comprising:
providing a reaction vessel, a circular plasmid with a known nucleotide sequence and a PCR amplified product of a linearized target DNA molecule with a known nucleotide sequence for encoding a desired target protein;
introducing into the reaction vessel at essentially the same time the circular plasmid and PCR amplified product of the linearized target DNA and at least two restriction enzymes into the reaction vessel in an amount to linearize the circular plasmid, wherein the restriction enzymes comprises a combination of BamHI and SalI;
adding to the reaction vessel an incubating solution, wherein the incubation solution comprises components comprising at least a DNA polymerase, a 5′-3′ exonuclease, a buffering solution comprising at least a crowding agent such as a PEG molecule, and other components including but not limited to dNTPs, a tris buffering agent, and optionally a DNA ligase;
incubating the components at a temperature and for a sufficient time for linearization of the circular plasmid and joining the PCR amplified product and a linearized circular plasmid for production of a circularized or linearized DNA molecule for subsequent expression in a host cell, wherein the incubation time and temperature is selected from the group 37° C. for 15 minutes +50° C. for about 15 minutes; 50° C. for 60 minutes; 37° C. for 15 minutes+50° C. for 45 minutes and 37° C. for 30 minutes +50° C. for 30 minutes.

In yet another aspect the present invention provides for a one pot method to prepare a circular or linear DNA molecule, the method comprising:

providing a reaction vessel, a circular plasmid and a PCR amplified product of a linearized target DNA molecule encoding a desired target protein;
introducing into the reaction vessel at essentially the same time the circular plasmid and PCR amplified product of the linearized target DNA and at least one restriction enzymes into the reaction vessel in an amount to linearize the circular plasmid;

adding to the reaction vessel a buffering solution, wherein the buffering solution comprises at least a DNA polymerase, a 5′-3′ exonuclease, a buffering agent and optionally a DNA ligase;

incubating the circular plasmid, the PCR amplified product of a linearized target DNA molecule and buffering solution for a sufficient time and temperature for linearization of the circular plasmid and
joining the PCR amplified product and the linearized circular plasmid for production of a circularized or linearized DNA molecule for subsequent expression in a host cell.

In a still further aspect, the present invention provides for a composition for a one pot synthesis of a circularized or linearized DNA molecule, the composition comprising;
a circular plasmid, a linearized target DNA molecule, at least one restriction enzyme, and preferably two restriction enzymes, at least a DNA polymerase, a 5′-3′ exonuclease, an incubating solution comprising a PEG molecule as a crowding agent, and other components including but not limited to dNTPs, a tris buffering agent, and optionally a DNA ligase. The linearized DNA target molecule can be amplified for inclusion in the composition.

In yet another aspect, the present invention also provides kits suitable for directionally cloning a linearized DNA target product into a circular DNA vector. The kit may comprise, in separate containers for adding to a single reaction vessel, an aliquot of a DNA polymerase having intrinsic exonuclease activity that is capable of performing the DNA joining reaction of the amplified products into a circular DNA vector, at least one restriction enzyme that may be in a separate container for adding to the reaction vessel and an aliquot of reaction buffer. An aliquot refers to an amount of the component sufficient to perform at least one program of cloning. The DNA polymerase may be provided as a solution of known concentration such a buffer solution that include other reagents wherein such reagents may include, together or in separate containers, PEG molecule as a crowding agent, and other components including but not limited to dNTPs, a tris buffering agent, and optionally a Taq ligase.

Various other aspects, features and embodiments of the invention will be more fully apparent from the ensuing disclosure and appended claims.

### BRIEF DESCRIPTION OF THE FIGURES

FIG. 1 shows the process for assembling a gene into a vector used by Prior Art Methods.

FIG. 2 shows the one-step assembly of circular vector DNA with target DNA of the present invention.

FIG. 3 shows the colony PCR screen for clones with the correct insert.

FIG. 4 shows a schematic of the invention with homology stitching oligos.

### DETAILED DESCRIPTION OF THE INVENTION

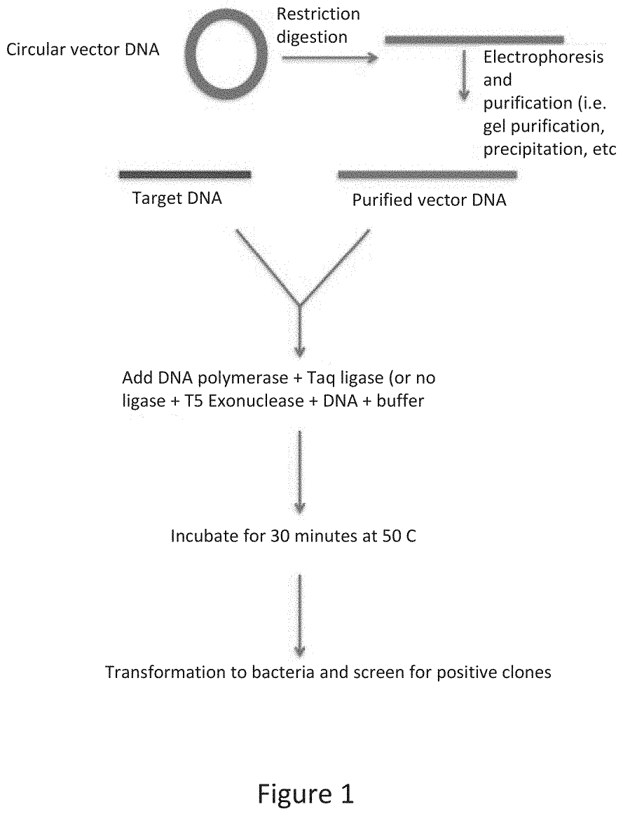

The present invention has multiple advantages over the existing methods. The first advantage is that it is less time consuming. The prior art method used pre-linearized DNA circular vectors and such restriction digestion of the vector DNA often takes 20 minutes to one hour. Notably restriction enzyme digestion of vector DNA is often incomplete as shown in FIG. 1. Trace amounts of incompletely digested vector DNA creates false positive clones that do not contain the target DNA molecule. It is technically extremely difficult to achieve successful assembly using the Gibson method if the vector is digested with a single restriction enzyme due to the rapid and preferential intramolecular re-ligation of the vector to itself during the assembly reaction. This is especially a problem when the assembly reaction is inefficient, for example, when joining multiple fragments is required to create the correct insert.

Then the next step in the prior art often entails requires electrophoresis of the DNA which may take at least one hour including the time to prepare the agarose gel. Purifying the prior art prelinearized vector DNA from the gel often takes more than 30 minutes. Other methods include precipitation and resuspending after restriction digestion.

Importantly, the present invention avoids all the above described time consuming steps and more importantly the high costs of such steps, such as the cost of agarose gel electrophoresis and the reagents for gel purification of vector DNA. Further, the method of the present invention makes it easier to achieve a high concentration of vector DNA in the reaction by eliminating the dilution and re-concentration steps necessitated by prior restriction digestion, gel electrophoresis and gel purification. High vector concentration is desirable since it markedly increases the number of positive clones obtained when the assembled DNA is transformed into bacteria cells.

The largest advantage of the present invention is lower background which is achieved by the continuous presence of the restriction enzyme in the novel and inventive system thereby allowing the dynamic digestion of self-ligated vector and greatly reducing the background.

As used herein, the term “5′-3′ exonuclease”, refers to an exonuclease that degrades DNA from the 5′ end, i.e., in the 5′ to 3′ direction. 5′-3′ exonucleases of interest can remove nucleotides from the 5′ end of a strand of ds DNA at a blunt end and, in certain embodiments, at a 3′ and or 5′ overhang. T5 exonuclease, lambda exonuclease and T7 exonuclease are examples of 5′-3′ exonucleases. In certain embodiments, T5 exonuclease is preferred. T5 exonuclease additionally has a ss endonuclease activity.

As used herein, the term “ligase”, refers to an enzyme that can covalently join a 3′ end of a DNA molecule to a 5′ end of another DNA molecule, particularly at a nick. Examples of ligases include T7 ligase, T4 DNA ligase, E. coli DNA ligase and Taq ligase, although many others are known and may be used herein.

As used herein, the term “overlapping sequence”, refers to a sequence that is complementary in two polynucleotides and where the overlapping sequence is ss, on one polynucleotide it can be hybridized to another overlapping complementary ss region on another polynucleotide.

As used herein the term “overhang” refers to the single stranded region of ds DNA at the end thereof and is either of type 5′ or 3′ due to the inherent directionality of DNA. The overhangs are generally generated in various lengths by treating dsDNA with restriction enzymes or exonucleases and/or by the addition of appropriate dNTPs (dATP, dTTP, dCTP, dGTP).

As used herein, the term “single strand (ss) DNA binding protein”, refers to proteins that bind to ss DNA and prevent premature annealing, protect the ss DNA from being digested by nucleases, and polymerases and/or remove secondary structure from the DNA to allow other enzymes to function effectively upon it. Inclusion of a ss binding protein in the compositions described herein is preferable to optimize the efficiency of synthon formation. Examples of ss DNA binding proteins are T4 gene 32 protein, E. coli SSB, T7 gp2.5 SSB, and phage phi29 SSB, and ET SSB although many others, e.g., RedB of lambda phage, RecT of Rac prophage and the sequences listed below, are known and may be used herein.

In a ligase-independent method of joining two ends of ds DNAs, it is important that 5′ or 3′ overhangs with optimal length are generated, which is done using a DNA polymerase having 3′->5′ exonuclease activity or 5′->3′ exonuclease respectively.

As used herein the term double stranded DNA (dsDNA) refers to oligonucleotides or polynucleotides having 3′ overhang, 5′ overhang or blunt ends and composed of two single strands all or part of which are complementary to each other, and thus dsDNA may contain a single stranded region at the ends and may be synthetic or natural origin derived from cells or tissues. In one embodiment, dsDNA is a product of PCR (Polymerase Chain Reaction) or fragments generated from genomic DNA or plasmids or vectors by a physical or enzyme treatment thereof.

As used herein, the term “buffering agent”, refers to an agent that allows a solution to resist changes in pH when acid or alkali is added to the solution. Examples of suitable non-naturally occurring buffering agents that may be used in the compositions, kits, and methods of the present invention include, for example, Tris, HEPES, TAPS, MOPS, tricine, or MES.

As used herein, the term “polynucleotide” encompasses oligonucleotides and refers to a nucleic acid of any length. Polynucleotides may be DNA or RNA. Polynucleotides may be ss or ds unless specified. Polynucleotides may be synthetic, for example, synthesized in a DNA synthesizer, or naturally occurring, for example, extracted from a natural source, or derived from cloned or amplified material. Polynucleotides referred to herein may contain modified bases.

The target nucleic acids utilized herein can be any nucleic acid, for example, human nucleic acids, bacterial nucleic acids, or viral nucleic acids. The target nucleic acid sample can be, for example, a nucleic acid sample from one or more cells, tissues, or bodily fluids such as blood, urine, semen, lymphatic fluid, cerebrospinal fluid, or amniotic fluid, or other biological samples, such as tissue culture cells, buccal swabs, mouthwashes, stool, tissues slices, biopsy aspiration, and archeological samples such as bone or mummified tissue. Target nucleic acids can be, for example, DNA, RNA, or the DNA product of RNA subjected to reverse transcription. Target samples can be derived from any source including, but not limited to, eukaryotes, plants, animals, vertebrates, fish, mammals, humans, non-humans, bacteria, microbes, viruses, biological sources, serum, plasma, blood, urine, semen, lymphatic fluid, cerebrospinal fluid, amniotic fluid, biopsies, needle aspiration biopsies, cancers, tumors, tissues, cells, cell lysates, crude cell lysates, tissue lysates, tissue culture cells, buccal swabs, mouthwashes, stool, mummified tissue, forensic sources, autopsies, archeological sources, infections, nosocomial infections, production sources, drug preparations, biological molecule productions, protein preparations, lipid preparations, carbohydrate preparations, inanimate objects, air, soil, sap, metal, fossils, excavated materials, and/or other terrestrial or extra-terrestrial materials and sources.

The sample may also contain mixtures of material from one source or different sources. For example, nucleic acids of an infecting bacterium or virus can be amplified along with human nucleic acids when nucleic acids from such infected cells or tissues are amplified using the disclosed methods. Types of useful target samples include eukaryotic samples, plant samples, animal samples, vertebrate samples, fish samples, mammalian samples, human samples, non-human samples, bacterial samples, microbial samples, viral samples, biological samples, serum samples, plasma samples, blood samples, urine samples, semen samples, lymphatic fluid samples, cerebrospinal fluid samples, amniotic fluid samples, biopsy samples, needle aspiration biopsy samples, cancer samples, tumor samples, tissue samples, cell samples, cell lysate samples, crude cell lysate samples, tissue lysate samples, tissue culture cell samples, buccal swab samples, mouthwash samples, stool samples, mummified tissue samples, autopsy samples, archeological samples, infection samples, nosocomial infection samples, production samples, drug preparation samples, biological molecule production samples, protein preparation samples, lipid preparation samples, carbohydrate preparation samples, inanimate object samples, air samples, soil samples, sap samples, metal samples, fossil samples, excavated material samples, and/or other terrestrial or extra-terrestrial samples. Types of forensics samples include blood, dried blood, bloodstains, buccal swabs, fingerprints, touch samples (e.g., epithelial cells left on the lip of a drinking glass, the inner rim of a baseball cap, or cigarette butts), chewing gum, gastric contents, saliva, nail scrapings, soil, sexual assault samples, hair, bone, skin, and solid tissue. Types of environmental samples include unfiltered and filtered air and water, soil, swab samples from surfaces, envelopes, and powders.

As used herein, the term “overlapping sequence”, refers to a sequence that is complementary in two polynucleotides and where the overlapping sequence is ss, on one polynucleotide it can be hybridized to another overlapping complementary ss region on another polynucleotide. By way of example, the overlapping sequence may be complementary in at least 5, 10, 15, or more polynucleotides in a set of polynucleotides. An overlapping sequence may vary in length and, in some cases, may be at least 12 nucleotides in length (e.g. at least 15, 20 or more nucleotides in length) and/or may be up 100 nucleotides in length (e.g., up to 50, up to 30, up to 20 or up to 15 nucleotides in length).

As used herein, the term “polynucleotide assembly”, refers to a reaction in which two or more, four or more, six or more, eight or more, ten or more, 12 or more 15 or more polynucleotides, e.g., four or more polynucleotides are joined to another to make a longer polynucleotide. The product of a polynucleotide assembly reaction, i.e., the “assembled polynucleotide” in many embodiments should contain one copy of each of the overlapping sequences.

As used herein, the term “incubating under suitable reaction conditions”, refers to maintaining a reaction a suitable temperature and time to achieve the desired results, i.e., polynucleotide assembly. Reaction conditions suitable for the enzymes and reagents used in the present method are described herein and, as such, suitable reaction conditions for the present method can be readily determined. These reactions conditions may change depending on the enzymes used (e.g., depending on their optimum temperatures, etc.).

As used herein, the term “Phusion polymerase” refers to thermal stable DNA polymerase that contains a Pyrococcus-like enzyme fused with a processivity-enhancing domain, resulting in increased fidelity and speed, e.g., with an error rate >50-fold lower than that of Tag DNA Polymerase and 6-fold lower than that of Pyrococcus furiosus DNA Polymerase. It possesses 5′-3′ polymerase activity and an example of Phusion polymerase is Phusion®. High-Fidelity DNA Polymerase (New England Biolabs).

As used herein, the term “joining”, refers to the production of covalent linkage between two sequences.

As used herein, the term “primer” as used herein refers to a bipartite primer or a primer having a first and second portion. A first portion of the primer is designed to be complementary to the appropriate end of a target DNA molecule and a second portion of the primer is designed to be complementary to nucleotide sequences on one side of the chosen restriction site of the circular plasmid, once linearized in the buffer solution of the present invention. Bipartite primers will generally have a minimum length of about 10 nucleotides and a maximum length of about 200 nucleotides and preferably about from 20 nucleotides to about 100 nucleotides, more preferably from about 30 nucleotides and about 40 nucleotides.

As used herein, the term “composition” refers to a combination of reagents that may contain other reagents, e.g., glycerol, salt, dNTPs, etc., in addition to those listed. A composition may be in any form, e.g., aqueous or lyophilized, and may be at any state (e.g., frozen or in liquid form).

Any one or more of the proteins (e.g., the ligase, SSBP, 5′-3′ exonuclease or polymerase, etc.) used herein may be temperature sensitive or thermostable where, as used herein, the term “temperature sensitive” refers to an enzyme that loses at least 95% of its activity after 10 minutes at a temperature of 65° C., and the term “thermostable” refers to an enzyme that retains at least 95% of its activity after 10 minutes at a temperature of 65° C.

The steps of the invention initially include attaching a primer to the linearized target nucleotide molecule. The linear target nucleotide molecule can be amplified by using the polymerase chain reaction with a first and second primers to provide a PCR amplified product. The 3′ end of the first primer molecule is designed to hybridize with the first end of the target DNA molecule, and the 5′ end of the first primer molecule has a sequence designed to incorporate sequences in the final PCR product that are complementary to the first end of the linearized plasmid DNA molecule after the circular plasmid interacts with a appropriate restriction enzyme to cut it at the chosen insert site. The 3′ end of the second primer is designed to hybridize with the second end of the target DNA molecule, and the 5′ end of the second primer molecule has a sequence designed to incorporate sequences in the final PCR product that are complementary to the second end of the linearized plasmid DNA molecule after interaction with the appropriate restriction enzyme. The two primers are then annealed to the target DNA molecule which is then PCR amplified using standard conditions to generate a PCR amplified product.

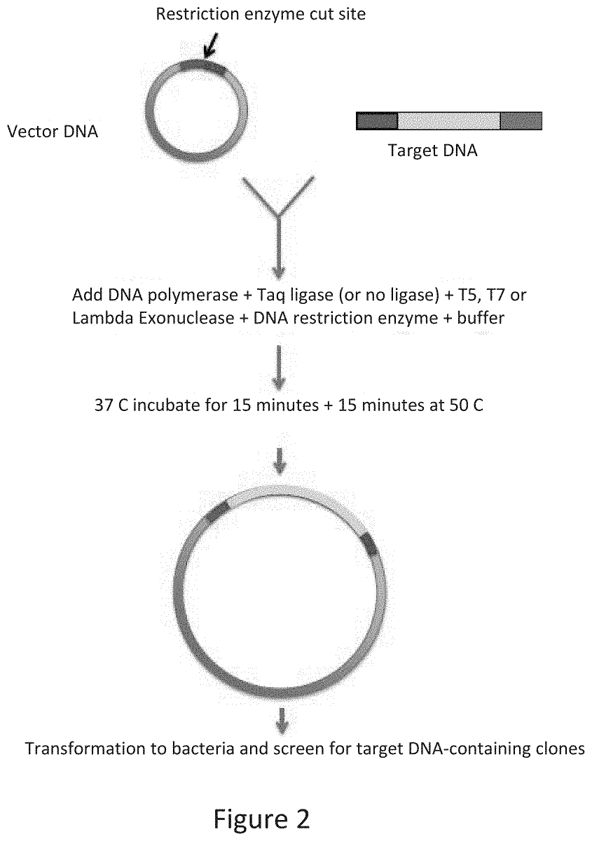

As shown in FIG. 2, the PCR amplified product is then simultaneously incubated with the circular plasmid in the presence of the appropriate restriction enzymes to cut it at the chosen insert sites using standard conditions, a suitable reaction buffer and in the presence of a DNA polymerase that is capable of performing the DNA joining reaction of the invention, for about 5 to about 60 minutes, preferably from about 10 to about 40 minutes, most preferably from about 15 to about 30 minutes. The reaction buffer may be any buffer that is used in DNA annealing reactions. The temperature may be in the range of from about 35-40° C., more preferably about 37° C.

The method of the invention may be used to clone any variety or number of target DNA molecules. The only limitation on size is the capacity of the circular DNA vector to carry the insert in transformation and replication in the host cell. Any circular DNA vector capable of replicating in a prokaryotic or eukaryotic cell is usable with the present invention. The choice of circular DNA vector, such as capsid, cosmid or bacterial artificial chromosome depends on the functional properties desired, for example, protein expression, and the host cell to be transformed. Preferably, the circular DNA vector has a known sequence of about 5 to about 100, preferably about 8 to about 50, most preferably about 10 to about 35 nucleotides, on either side of the chosen restriction enzyme site.

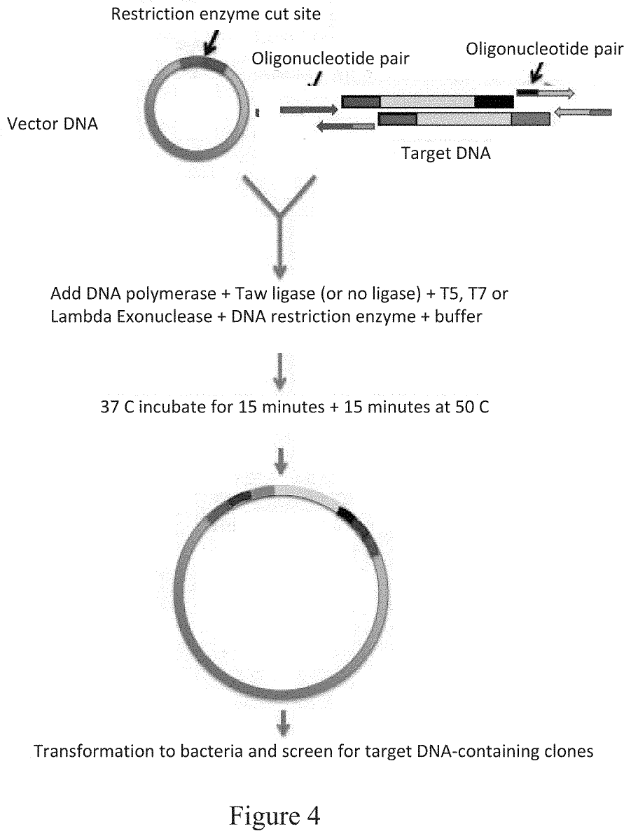

In another embodiment, pairs of single stranded oligonucleotides can be used to generate a region of sequence overlap between the vector and the target DNA molecule or between two target DNA molecules that includes additional nucleotides not found in the vector or the target DNA, e.g. when it is desirable to add a promoter sequence or the DNA sequence encoding a tag for a protein such as shown in FIG. 4

Transformation of Recombinant DNA Molecules

Any circular plasmid may be used [4]. Typical expression circular plasmids contain a promoter, an enhancer, a coding sequence and a terminator. The promoter region of the plasmid binds RNA polymerase II, associated enzymes and other factors, which are required to initiate transcription. The function of enhancer sequences is to bind specific intracellular transcription factors. The DNA-bound transcription factors interact with the transcription complex and increased the transcription rate. Normal endogenous transcription factors are proteins that contain two domains, the DNA binding domain and the transcription activation domain. The DNA binding domain binds to specific duplex DNA sequences, usually 5-10 base pairs, located in the enhancer region. The DNA binding domain brings the transcription activation domain into proximity of the minimal promoter where it interacts with RNA polymerase to activate transcription.

The present examples utilized a commercially available plasmid with a selectable marker. Any selectable marker may be used. Similarly a specific recognition site for any restriction cleavage enzyme capable of specifically cleaving at the ends of the oligonucleotide to generate either staggered ends or blunt ends may be selected where the specific cleavage site does not occur in the fragments of interest in addition to the engineered position adjacent to the ends of the fragment of interest. In the present invention, the recognition site for the restriction enzyme that produces staggered ends has been introduced adjacent to the polynucleotide of interest by means of DNA synthesis.

The reaction mixture obtained from the incubation of DNA polymerase and restriction enzyme with the circular plasmid and the PCR amplified product may be used to transform any host cell using standard transformation procedures. Such hosts can be, in particular, bacteria or eukaryotic cells (yeasts, animal cells, plant cells), and the like. Among bacteria, Escherichia coli, Bacillus subtilis, Streptomyces, Pseudomonas (P. putida, P. aeruginosa), Rhizobium meliloti, Agrobacterium tumefaciens, Staphylococcus aureus, Streptomyces pristinaespirais, Enterococcus faeciumor Clostridium, and the like, may be mentioned. Among bacteria, E. coli is commonly used. Among yeasts, Kluyveromyces, Saccharomyces, Pichia, Hansenula, and the like, may be mentioned. Among mammalian animal cells, CHO, COS, NIH3T3, and the like, may be mentioned.

In accordance with the host used, a person skilled in the art will adapt the selection/replication of plasmid described in the invention. In particular, the origin of replication and the selection marker gene are chosen in accordance with the host cell selected.

The selection marker gene may be a resistance gene, for example, conferring resistance to an antibiotic (ampicillin, kanamycin, geneticin, hygromycin, and the like), or any gene endowing the cell with a function, which it no longer possesses (for example, a gene which has been deleted on the chromosome or rendered inactive), the gene on the plasmid reestablishing this function. This selectable marker gene allows plasmid selection and production in minimal media.

The present invention will be further illustrated in the following example. However, it is to be understood that this example is for illustrative purposes only and should not be used to limit the scope of the present invention in any manner.

### EXAMPLE 1

Cloning the human SRSF3 gene

Although many methods have been developed to assemble linear DNA molecules, a method to assemble circular DNA to circular DNA or circular DNA to linear DNA in one step has not been developed. Here the inventors describe a method to directly assemble a circular plasmid DNA with a linear PCR product in one step.

The circular DNA is a plasmid which uses the vector pQE80L as a backbone and contains human RPS6 gene. The linear PCR product is human SRSF3, and the primers used to amplify SRSF3 genes are: GCATCACCATCACCATCACGtgcatcgtgattcctgtcc (SEQ ID NO: 1) and TAATTAAGCTTGGCTGCAGGctatttcctttcatttgacc (SEQ ID NO 2). Each primer has a 20-base pair homology with the vector. SRSF3 gene is amplified using HeLa cell cDNA as template. To assemble the linear PCR product, 100 ng of plasmid DNA is mixed with 300 ng of PCR product and mixed with 1 ul of BamHI and 1 ul of SalI together with a buffer plus Phusion Taq polymerase, Taq ligase and T5 exonuclease.

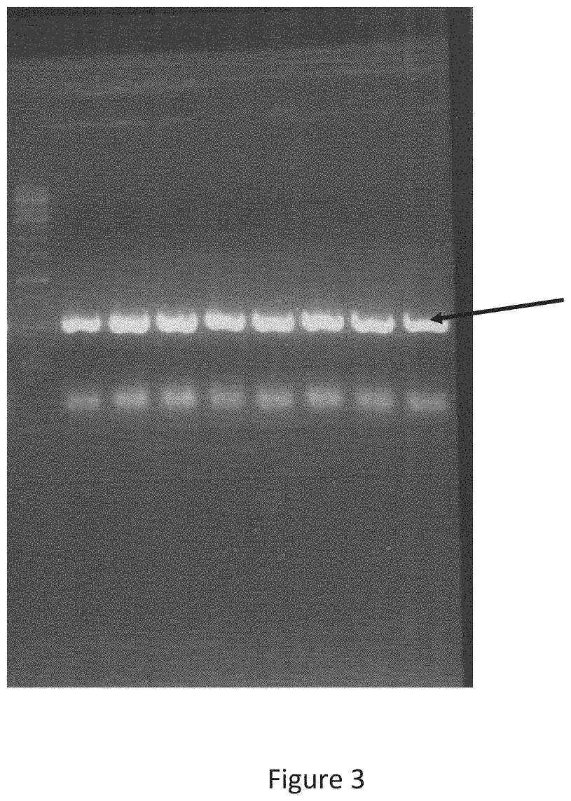

The mixture is incubated at 37° C. for 15 minutes then 50° C. for 45 minutes to assemble SRSF3 into pQE80L. The reaction mixture is passed through a column to remove salts and the DNA was used to transform E. coli DH10B competent cells. The colonies are screened with colony PCR. 8 colonies were picked from the plates and screened with colony PCR using the same primers. Plasmids purified from all 8 colonies contain the correct insert and shown in FIG. 3. Arrow shows correct sized insert.

### REFERENCES

The references cited below are incorporated by reference herein for all purposes.

U.S. Pat. No. 7,723,077

U.S. Pat. No. 7,575,860

Yongzhen, Xia et al., T5 exonuclease-dependent assembly offers a low-cost method for efficient cloning and site-directed mutagenesis, Feb. 2019, Nucleic Acids Research, V. 47, Issue 3, Page 15.

Masaki Shintani, et al.. 2015, Genomics of microbial plasmids: classification and identification based on replication and transfer systems and host taxonomy, Front. Microbiol., 31 March 2015|https://doi.org/10.3389/fmicb.2015.00242.

1. A one pot method to prepare a circular or linear DNA molecule for use in preparing a nucleotide end-product, the method comprising:
providing a reaction vessel, a combination of a circular DNA vector and an amplified linearized target DNA molecule;
introducing into the reaction vessel at essentially the same time the circular DNA vector and the amplified linearized target DNA molecule and at least one restriction enzyme into the reaction vessel in an amount to linearize the circular DNA vector;
adding to the reaction vessel a buffering solution, wherein the buffering solution comprises at least a DNA polymerase, a 5′-3′ exonuclease, a buffering agent and optionally a DNA ligase;
incubating the circular DNA vector, the amplified linearized target DNA molecule and the buffering solution for a sufficient time and temperature for linearization of the circular DNA vector and joining the amplified linearized target DNA molecule and the linearized circular DNA vector for production of the circularized or linearized DNA molecule.

2. The method of claim 1, comprising a DNA ligase and wherein the DNA ligase is selected from the group consisting of Taq DNA ligase; 9N DNA ligase and Ampligase.

3. The method of claim 1, wherein the DNA polymerase enzyme is selected from the group consisting of a Phusion DNA polymerase, platinum Taq DNA polymerase High Fidelity, and Pfu DNA polymerase.

4. The method of claim 1, wherein the buffering solution comprises at least a crowding agent, dNTPs, potassium acetate, magnesium acetate, bovine serum albumin, and a tris acetate buffering agent.

5. The method of claim 1, wherein the 5′-3′ exonuclease is selected from the group consisting of T5 exonuclease, lambda exonuclease and T7 exonuclease.

6. The method of claim 4, wherein the crowding agent is a PEG molecule.

7. The method of claim 1, wherein the buffering solution comprises 2% to 10% of PEG8000, 50 mM to about 150 mM of Tris-Acetate, pH 6.5 to 8.5, from about 0.09 mM to about 0.4 mM dNTPs, from about 25 mM to about 75 mM of potassium acetate, about 10 mM to about 40 mM of magnesium acetate and about 50 mg/ml to about 150 mg/ml Bovine Serum Albumin.

8. The method of claim 1, wherein the sufficient time and temperature for incubation is selected from the group consisting of 37° C. for 15 minutes +50° C. for about 15 minutes; 37° C. for 15 minutes+50° C. for 45 minutes and 37° C. for 30 minutes +50° C. for 30 minutes.

9. The method of claim 1, wherein the at least one restriction enzyme is a combination of BamHI and SalI.

10. The method of claim 1, wherein the nucleotide end-product is selected from the group consisting of double stranded DNA, circular or linear DNA molecule, circular or linear RNA molecule or a protein encoded by the circularized or linearized DNA molecule through host cell production.

11. A one pot method to prepare a circular or linear DNA molecule, the method comprising:
providing a reaction vessel, a circular plasmid and a PCR amplified product of a linearized target DNA molecule encoding a desired target protein;
introducing into the reaction vessel at essentially the same time the circular plasmid and PCR amplified product of the linearized target DNA and at least two restriction enzymes into the reaction vessel in an amount to linearize the circular plasmid, wherein the restriction enzymes comprises a combination of BamHI and SalI;
adding to the reaction vessel an incubating solution, wherein the incubation solution comprises components comprising at least a DNA polymerase, a 5′-3′ exonuclease, a buffering solution comprising at least a crowding agent such as a PEG molecule, dNTPs, potassium acetate, magnesium acetate, a tris acetate buffering agent, bovine serum albumin, and optionally a DNA ligase;
incubating the components at a temperature and for a sufficient time for linearization of the circular plasmid and joining the PCR amplified product and the linearized circular plasmid for production of a circularized or linearized DNA molecule for subsequent expression in a host cell, wherein the incubation time and temperature is selected from the group 37° C. for 15 minutes +50° C. for about 15 minutes; 50° C. for 60 minutes; 37° C. for 15 minutes+50° C. for 45 minutes and 37° C. for 30 minutes +50° C. for 30 minutes.

12. The method of claim 11, wherein the DNA ligase is added and selected from the group consisting of Taq DNA ligase; 9N DNA ligase and Ampligase.

13. The method of claim 11, wherein the DNA polymerase enzyme is selected from the group consisting of a Phusion DNA polymerase, platinum Taq DNA polymerase High Fidelity, and Pfu DNA polymerase.

14. The method of claim 11, wherein the 5′-3′ exonuclease is selected from the group consisting of T5 exonuclease, lambda exonuclease and T7 exonuclease.

15. A composition for a one pot synthesis of a circularized or linearized DNA molecule, the composition comprising;
a circular DNA vector, a linear DNA molecule, at least one restriction enzyme. at least a DNA polymerase, a 5′-3′ exonuclease, an incubating solution comprising at least a crowding agent, dNTPs, and a tris buffering agent, and optionally a DNA ligase.

16. The composition of claim 15, comprising a DNA ligase and wherein the DNA ligase is selected from the group consisting of Taq DNA ligase; 9N DNA ligase and Ampligase.

17. The composition of claim 15, wherein the DNA polymerase enzyme is selected from the group consisting of a Phusion DNA polymerase, platinum Taq DNA polymerase High Fidelity, and Pfu DNA polymerase.

18. The composition of claim 15, wherein the 5′-3′ exonuclease is selected from the group consisting of T5 exonuclease, lambda exonuclease and T7 exonuclease.

19. The composition of claim 15, wherein the crowding agent is a PEG molecule.

20. The composition of claim 15, wherein the incubation solution comprises 2% to 10% of PEG8000, 50 mM to about 150 mM of Tris-Acetate, pH 6.5 to 8.5, from about 0.09 mM to about 0.4 mM dNTPs, from about 25 mM to about 75 mM of potassium acetate, about 10 mM to about 40 mM of magnesium acetate and about 50 mg/ml to about 150 mg/ml Bovine Serum Albumin.


SEQUENCE LISTING (2 sequences)

SEQ ID NO: 1 - DNA, 39 bp, Artificial Sequence
  Synthetic Construct
gcatcaccatcaccatcacgtgcatcgtgattcctgtcc

SEQ ID NO: 2 - DNA, 40 bp, Artificial Sequence
  Synthetic Construct
taattaagcttggctgcaggctatttcctttcatttgacc